(19) United States
(12) Patent Application Publication
(10) Pub. No.: US 2013/0210879 A1
(43) Pub. Date: Aug. 15, 2013

(54) FORMULATIONS OF BENDAMUSTINE

(71) Applicant: Eagle Pharmaceuticals, Inc., (US)

(72) Inventors: Nagesh R. Palepu, Southampton, PA (US); Philip Christopher Buxton, Great Dunmow (GB); Srikanth Sundaram, Somerset, NJ (US)

(73) Assignee: EAGLE PHARMACEUTICALS, INC., Woodcliff Lake, NJ (US)

(21) Appl. No.: 13/767,672

(22) Filed: Feb. 14, 2013

Related U.S. Application Data
(60) Provisional application No. 61/598,729, filed on Feb. 14, 2012.

(51) Int. Cl. A61K 47/10; A61K 31/4184

(52) U.S. Cl. CPC A61K 47/10; A61K 31/4184
USPC 514/394

(57) ABSTRACT

Long term storage stable bendamustine-containing compositions are disclosed. The compositions can include bendamustine or a pharmaceutically acceptable salt thereof, and a pharmaceutically acceptable fluid contains a mixture of PEG and PG; an organic or inorganic compound in an amount sufficient to obtain a pH of from about 6.0 to about 11 for the polyethylene glycol, as measured using USP monograph for polyethylene glycol; and optionally an antioxidant. The bendamustine-containing compositions have less than about 5% total esters, on a normalized peak area response (“PAR”) basis as determined by high performance liquid chromatography (“HPLC”) at a wavelength of 223 nm, after at least about 15 months of storage at a temperature of from about 5° C. to about 25° C.

### CROSS-REFERENCE TO RELATED APPLICATION

This patent application claims priority under 35 U.S.C. §119(e) to U.S. Provisional Patent Application No. 61/598,729, filed Feb. 14, 2012, entitled “FORMULATIONS OF BENDAMUSTINE”, the contents of which are incorporated by reference herein in its entirety.

### BACKGROUND OF THE INVENTION

Bendamustine free base is represented by the following structural formula (I)

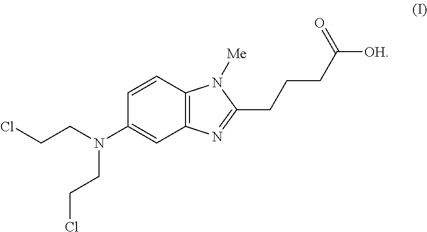 [SMILES: Cn1c(CCCC(=O)O)nc2cc(N(CCCl)CCCl)ccc21]

Bendamustine is used in the treatment of a number of cancers including leukemias, Hodgkin's disease and multiple myelomas. Bendamustine is the active ingredient of the commercial product Treanda™, a lyophilized powder for reconstitution.

Bendamustine exhibits rapid degradation upon reconstitution of the lyophilized product. Bendamustine undergoes hydrolysis by direct substitution rather than an addition elimination process due to the presence of the highly labile aliphatic chlorine atoms. Some of the main degradants of bendamustine are the monohydroxy compound known as HP1 (hydrolysis product 1) and dihydroxy compound HP2 (hydrolysis product 2). The monohydroxy compound appears as the main impurity at Relative Retention Time (RRT) 0.6 and the dihydroxy compound appears as the main impurity at RRT 0.27. Minor peaks appear at RRT 1.2, which are presently unknown.

The stability of bendamustine in water is measured in hours, and is therefore, not suitable for long-term storage in liquid form. The lyophile possesses good chemical stability. However, reconstitution of the lyophile is clinically inconvenient, taking 15-30 minutes with implications of chemical instability. There is a need for ready to use (RTU) bendamustine formulations having enhanced stability.

Some parenteral formulations containing lower molecular weight PEG's have significant variations in long term product stability from batch to batch. It has been determined that at least some and perhaps all of this unacceptable property is attributable to the PEG included therein. The amount of degradation observed in such formulations, typically in the form of PEG-esters of bendamustine, negatively impacts the expected shelf life of the formulations. Reproducibility of batch to batch stability assures consistent product potency and reduces the need for premature product recall and destruction.

Lower molecular weight polyethylene glycols (PEG's) such as liquid PEG's having molecular weights from 200 to 600, PEG 400 most commonly, have been included in pharmaceutical formulations for decades. They are available from a number of suppliers globally. It has been found that there is significant variability in the excipient's stability depending upon the supplier, storage conditions, handling conditions, etc. Sometimes, batches including the PEG as received from the supplier have the performance specifications expected. Other times, they do not. This even occurs in some situations when an initial batch made with a certain supplier's PEG met performance requirements. Preventing or counteracting the deleterious of effects of some PEG's in liquid formulations would be an advance in the art. The present invention addresses this need.

### SUMMARY OF THE INVENTION

In some aspects of the invention, the liquid bendamustine-containing compositions include a) a pharmaceutically acceptable fluid which contains a mixture of propylene glycol and polyethylene glycol, b) an organic compound or an inorganic compound in an amount sufficient to obtain a pH of from about 6.0 to about 11 for the polyethylene glycol as measured using United States Pharmacopeia (USP) official monograph for polyethylene glycol, and c) a stabilizing amount of an antioxidant. The amount of bendamustine as calculated on the basis of the HCl salt included in the composition is preferably from about 20 mg/mL to about 60 mg/mL. Still further aspects of the invention include methods of treatment using bendamustine-containing compositions and kits containing the same.

One of the advantages of the inventive liquid compositions is that they have substantially improved long term stability. The batch to batch variability in stability attributable to the PEG included therein has been overcome. For example, the inventive bendamustine compositions are substantially free of impurities after at least about 15 months at a temperature of from about 5° C. to about 25° C. The inventive formulations are advantageously ready to use or ready for further dilution. Reconstitution of lyophilized powders when therapy is desired is not required.

### BRIEF DESCRIPTION OF THE DRAWINGS

FIGS. 1-8 are data tables corresponding to Examples 1-7.

FIG. 1 is data Table 1 corresponding to Comparative Example 1.

FIG. 2 is data Table 2 corresponding to Example 2.

FIG. 3 is data Table 3 corresponding to Example 3.

FIG. 4A is data Table 4A corresponding to Example 4.

FIG. 4B is data Table 4B corresponding to Example 4.

FIG. 5A is data Table 5A corresponding to Example 5.

FIG. 5B is data Table 5B corresponding to Example 5.

FIG. 6 is data Table 6 corresponding to Example 6.

FIG. 7 is data Table 7 corresponding to Example 6.

FIG. 8 is data Table 8 corresponding to Example 7.

### DETAILED DESCRIPTION OF THE INVENTION

Unless defined otherwise, all technical and scientific terms used herein have the same meaning as is commonly understood by one of ordinary skill in the art to which this invention belongs. In the event that there is a plurality of definitions for a term herein, those in this section prevail unless stated otherwise.

As used herein, RRT is calculated by dividing the retention time of the peak of interest by the retention time of the main peak. Any peak with an RRT <1 elutes before the main peak, and any peak with an RRT >1 elutes after the main peak.

For purposes of the present invention, “substantially free of impurities” shall be understood to include bendamustine-containing compositions in which the amount of total polyethylene glycol esters and propylene glycol esters is less than about 5%, as calculated on a normalized peak area response (“PAR”) basis as determined by high performance liquid chromatography (“HPLC”) at a wavelength of 223 nm, after a period of about 15 months at a temperature of from about 5° C. to about 25° C. The amount of impurities is further calculated as being based upon the original amount bendamustine (or salt thereof) being present in the composition or formulation. In one embodiment, the amount of total impurities in the inventive compositions resulting from the degradation of the bendamustine as evidenced by e.g. PEG-bendamustine esters and PG esters thereof, is less than about 3%, and more preferably less than about 2.4%, PAR as determined by HPLC at a wavelength of 223 nm after at least about 2 years at a temperature of from about 5° C. to about 25° C.

For purposes of the present invention, a pharmaceutically acceptable fluid is a fluid which is suitable for pharmaceutical use.

Preferably, in the inventive compositions, the amount of any individual polyethylene glycol esters does not exceed 0.2% and the amount of any individual propylene glycol esters does not exceed 1.5% PAR as determined by HPLC at a wavelength of 223 nm after storage periods of at least about 15 months at a temperature of from about 5° C. to about 25° C. Preferably, the amount of total polyethylene glycol esters is less than about 2%. Preferably, the amount of total propylene glycol esters is less than about 3%. In some aspects, the amount of time the inventive compositions demonstrate long term storage stability is at least about 18 months and preferably at least about 2 years when stored under the conditions described herein.

In accordance with one aspect of the invention there are provided long term storage stable bendamustine-containing compositions including:
a) bendamustine or a pharmaceutically acceptable salt thereof; and
b) a pharmaceutically acceptable fluid including
   i) a mixture of PEG and PG;
   ii) an organic compound or inorganic compound, or mixtures thereof in an amount sufficient to obtain an apparent pH of from about 6.0 to about 11 for the polyethylene glycol, as measured using USP official monograph for polyethylene glycol; and
   iii) a stabilizing amount of an antioxidant.

The total impurities in the inventive compositions resulting from the degradation of the bendamustine in the compositions is less than about 5% PAR as determined by HPLC at a wavelength of 223 nm after at least about 15 months at a temperature of from about 5° C. to about 25° C., and thus have long term stability for at least the same period of time or longer. Preferably, the bendamustine-containing compositions demonstrate long term storage stability for at least about 2 years, especially when stored at the lower (refrigerated) temperatures.

In some aspects of the invention, the bendamustine is preferably present in the formulation as the HCl salt.

In some aspects of the invention, the bendamustine concentration calculated on the basis of the HCl salt in the inventive compositions is from about 10 mg/mL to about 100 mg/mL, preferably 20 mg/mL to about 60 mg/mL. Preferably the bendamustine concentration in the inventive compositions is from about 25 mg/mL to about 50 mg/mL, and more preferably from about 30 mg/mL to about 50 mg/mL. It will be understood that compositions containing any useful concentration within the ranges, i.e. 10, 20, 25, 30, 35, 40, 45, 50, 55, 60 . . . 100 are contemplated. In other embodiments, the bendamustine concentration in the composition is about 25 mg/mL. In alternative aspects, the amount of bendamustine is outside these ranges but the amounts will be sufficient for single or multiple administrations of dosages generally regarded as effective amounts.

In several embodiments of the invention, pharmaceutically acceptable fluid is non-aqueous and may be, but is not necessarily, a solvent for the bendamustine or salt thereof. Within this aspect, the pharmaceutically acceptable fluid is a mixture of propylene glycol (PG) and polyethylene glycol (PEG). For example, the pharmaceutically acceptable fluid can include about 50% PEG and about 50% PG. Alternatively, pharmaceutically acceptable fluid includes about 95% PEG and about 5% PG. The amount of PEG and PG can also be varied within the ranges, i.e. the ratio of PEG:PG in the pharmaceutically acceptable fluid can range from about 95:5 to about 50:50. Within this range, is a pharmaceutically acceptable fluid containing about 75% PEG and about 25% PG, and preferably 80% PEG and 20% PG. In another embodiment, a pharmaceutically acceptable fluid can include about 85% PEG and about 15% PG while another preferred pharmaceutically acceptable fluid includes about 90% PEG and about 10% PG. The molecular weight of the PEG is within the range of pharmaceutically acceptable weights although PEG 400 is preferred in many aspects of the invention.

In accordance with the USP official monograph for polyethylene glycol, see USP 35-NF30, the contents of which are incorporated by reference herein, the PEG pH is determined as follows: 5 g of PEG is dissolved into 100 ml carbon dioxide free water, and 0.3 ml of saturated KCl solution is added. The pH is then measured. This value is sometimes referred to as the apparent pH. Different amounts of organic or inorganic compounds can be added to the PEG in order to arrive at a pH of from about 6.0 to about 11. Preferably, the pH of the PEG is from about 6.0 to about 11. More preferably, the pH of the PEG is from about 6.5 to about 8. In other preferred aspects, the pH is about 8.

The pH of the PEG is not the same as the pH of the final bendamustine HCl formulation. Preferably, the pH of the final bendamustine-containing formulation is from about 3.3 to about 4. More preferably, the pH of the final bendamustine-containing formulation is about 3.5. The pH of the final bendamustine-containing formulation is measured in accordance with the USP official monograph for polyethylene glycol. Preferably, a 5 g aliquot of the final bendamustine-containing formulation is added to 100 ml carbon dioxide free water, and 0.3 ml of saturated KCl solution is added. The pH is then measured and adjusted if necessary to the preferred range.

Without meaning to be bound by any theory or hypothesis, polyethylene glycol quality can vary from batch to batch, manufacturer to manufacturer, over product lifetime and as a result of handling. Such variation has made it difficult to make reproducible long term storage stable bendamustine-containing formulations with high amounts of polyethylene glycol and propylene glycol, as the formation of PEG and PG esters is high. In order to obtain reproducible formulations, PEG is treated with an organic or inorganic compound to achieve the desired USP apparent pH. This treatment results in reproducible long-term storage stable bendamustine-containing compositions, with substantially no PEG or PG ester formation.

The bendamustine-containing compositions according to several preferred aspects of the invention include a stabilizing amount of an antioxidant. For purposes of the present invention, “stabilizing amount” shall be understood to include those amounts which increase or enhance the stability of the bendamustine in the compositions described herein. The presence of one or more antioxidants described herein thus contributes, at least in part to the long term stability of the composition. Within this guideline, suitable antioxidant concentrations in the compositions can range from about 2.5 mg/mL to about 35 mg/mL, and preferably from about 5 mg/mL to about 20 mg/mL or from about 10 mg/mL to about 15 mg/mL. In some other embodiments, the concentration of the antioxidant in the bendamustine-containing composition is about 5 mg/mL.

Suitable antioxidants for inclusion include those which are pharmaceutically acceptable for use in human and veterinary formulations although not limited to those currently regarded as safe by any regulatory authority. For example, the antioxidant can be selected from among lipoic acid, thioglycerol (also known as monothioglycerol) and analogs thereof, propyl gallate, methionine, cysteine, metabisulfites, sodium formaldehyde sulfoxylate, phenol-containing aromatic and aliphatic compounds, dihydrolipoic acid and mixtures of the foregoing. Preferably, the antioxidant is thioglycerol, lipoic acid or a mixture thereof. Some particularly preferred embodiments of the invention include thioglycerol.

In some aspects of the invention, organic compounds, inorganic compounds, and mixtures thereof are suitable acidity/alkalinity adjustors. Organic compounds include carboxylic compounds, nitrogenous compounds, carbonates, bicarbonates, and salts thereof. Preferably, the organic compounds are selected from monoethanolamine, diethanolamine, ethylenediaminetetraacetic acid (EDTA) phospholipid salts, ascorbate, ascorbic acid, sodium citrate, sodium sulfonic acid, sodium lauryl sulfate, quaternary amines, quaternary ammonium salts, and sodium acetate. Preferably, the organic compounds are selected from inorganic salts of organic acids. More preferably, the organic compound is sodium acetate. Inorganic compounds include compounds known to those of skill in the art, including, but not limited to, salts of hydroxides and salts of phosphates, sodium formate, sodium phosphate, potassium hydroxide, and phosphoric acid. Most preferably, the inorganic compound is sodium hydroxide.

In some embodiments of the invention, the amount of the organic compound or inorganic compound functioning as the acidity/alkalinity adjustor is provided in an amount sufficient to obtain a pH of from about 6.0 to about 11 for the polyethylene glycol, as measured using USP monograph for polyethylene glycol. In some aspects of the invention, about 0.5 μL to about 50 μL of a 1N acidity/alkalinity adjustor solution is provided per 1 mL of a bendamustine-containing composition. Preferably, about 1 μL to about 10 μL of a 1N acidity/alkalinity adjustor solution is provided per 1 mL of a bendamustine-containing composition. In some aspects of the invention, the acidity/alkalinity adjustor is added to the polyethylene glycol prior to the addition of the other materials in the formulation. In other aspects the acidity/alkalinity adjustor is added to the pharmaceutically acceptable fluid after the addition of the other materials to adjust the acidity or alkalinity as needed. In some aspects of the invention, the concentration of the organic compound in the final formulation is from about 0.005M (molarity) to about 0.1M (molarity), and more preferably, about 0.01M. In some aspects of the invention, the concentration of the inorganic compound in the final formulation is from about 0.0005M (molarity) to about 0.04M (molarity). It will be understood that any useful concentration within the ranges, i.e. 0.001, 0.0015, 0.005, 0.01, 0.02, 0.03, 0.04 are contemplated. Preferably, the concentration of the inorganic compound in the final formulation is about 0.01 molarity.

In view of the foregoing, some preferred non-aqueous, liquid, long term storage stable bendamustine-containing compositions in accordance with the invention include:
I. a) bendamustine or a pharmaceutically acceptable salt thereof; and
   b) a pharmaceutically acceptable fluid including
      i) polyethylene glycol and propylene glycol;
      ii) an organic compound or inorganic compound, or mixtures thereof in an amount sufficient to obtain a pH of from about 6.0 to about 11 for the polyethylene glycol, as measured using USP monograph for polyethylene glycol; and
      iii) a stabilizing amount of thioglycerol; or
II. a) about 25 mg/mL bendamustine or a pharmaceutically acceptable salt thereof; and
   b) a pharmaceutically acceptable fluid including
      i) about 90% PEG and about 10% PG;
      ii) an organic compound or inorganic compound, or mixtures thereof in an amount sufficient to obtain a pH of from about 6.0 to about 11 for the polyethylene glycol, as measured using USP monograph for polyethylene glycol; and
      iii) about 2.5 mg/mL thioglycerol.

Each of these compositions have the same stability profiles already described, i.e. having less than about 5% total esters, PAR as determined by HPLC at a wavelength of 223 nm, after at least about 15 months of storage at a temperature of from about 5° C. to about 25° C.

Some more preferred formulations include:

I. a) bendamustine or a pharmaceutically acceptable salt thereof; and
b) a pharmaceutically acceptable fluid including
   i) polyethylene glycol and propylene glycol;
   ii) sodium hydroxide in an amount sufficient to obtain a pH of from about 6.5 to about 11 for the polyethylene glycol, as measured using USP monograph for polyethylene glycol; and
   iii) a stabilizing amount of thioglycerol; or
      II. a) bendamustine or a pharmaceutically acceptable salt thereof at a concentration of about 25 mg/mL; and
b) a pharmaceutically acceptable fluid including
   i) 90% polyethylene glycol and 10% propylene glycol;
   ii) sodium hydroxide in an amount sufficient to obtain a pH of from about 6.5 to about 11 for the polyethylene glycol, as measured using USP monograph for polyethylene glycol; and
   iii) thioglycerol at a concentration of about 5 mg/mL; or
      III. a) bendamustine or a pharmaceutically acceptable salt thereof at a concentration of about 25 mg/mL; and
b) a pharmaceutically acceptable fluid including
   i) 85% polyethylene glycol and 15% propylene glycol;
   ii) sodium hydroxide in an amount sufficient to obtain a pH of from about 6.5 to about 11 for the polyethylene glycol, as measured using USP monograph for polyethylene glycol; and
   iii) thioglycerol at a concentration of about 5 mg/mL; or
      IV. a) bendamustine or a pharmaceutically acceptable salt thereof; and
b) a pharmaceutically acceptable fluid including
   i) polyethylene glycol and propylene glycol;
   ii) sodium acetate in an amount sufficient to obtain a pH of from about 6.5 to about 11 for the polyethylene glycol, as measured using USP monograph for polyethylene glycol; and
   iii) a stabilizing amount of thioglycerol; or
      V. a) bendamustine or a pharmaceutically acceptable salt thereof at a concentration of about 25 mg/mL; and
b) a pharmaceutically acceptable fluid including
   i) 90% polyethylene glycol and 10% propylene glycol;
   ii) sodium acetate in an amount sufficient to obtain a pH of from about 6.5 to about 11 for the polyethylene glycol, as measured using USP monograph for polyethylene glycol; and
   iii) thioglycerol at a concentration of about 5 mg/mL; or
      VI. a) bendamustine or a pharmaceutically acceptable salt thereof at a concentration of about 25 mg/mL; and
b) a pharmaceutically acceptable fluid including
   i) 85% polyethylene glycol and 15% propylene glycol;
   ii) sodium acetate in an amount sufficient to obtain a pH of from about 6.5 to about 11 for the polyethylene glycol, as measured using USP monograph for polyethylene glycol; and
   iii) thioglycerol at a concentration of about 5 mg/mL.

Each of these compositions have the same stability profiles already described, i.e. having less than about 5% total esters, on a normalized peak area response (“PAR”) basis as determined by high performance liquid chromatography (“HPLC”) at a wavelength of 223 nm, after at least about 15 months of storage at a temperature of from about 5° C. to about 25° C.

In other aspects of the invention, preferred long term storage stable bendamustine-containing compositions in accordance with the invention include:

I. a) bendamustine or a pharmaceutically acceptable salt thereof; and
b) a pharmaceutically acceptable fluid including
   i) polyethylene glycol and propylene glycol;
   ii) sodium hydroxide in an amount sufficient to obtain a pH of from about 3.3 to about 4.2 for the long term storage stable bendamustine-containing composition, as measured using USP monograph for polyethylene glycol; and
   iii) a stabilizing amount of thioglycerol; or
      II. a) bendamustine or a pharmaceutically acceptable salt thereof at a concentration of about 25 mg/mL; and
b) a pharmaceutically acceptable fluid including
   i) 90% polyethylene glycol and 10% propylene glycol;
   ii) sodium hydroxide in an amount sufficient to obtain a pH of from about 3.3 to about 4.2 for the long term storage stable bendamustine-containing composition, as measured using USP monograph for polyethylene glycol; and
   iii) thioglycerol at a concentration of about 5 mg/mL; or
      III. a) bendamustine or a pharmaceutically acceptable salt thereof at a concentration of about 25 mg/mL; and
b) a pharmaceutically acceptable fluid including
   i) 85% polyethylene glycol and 15% propylene glycol;
   ii) sodium hydroxide in an amount sufficient to obtain a pH of from about 3.3 to about 4.2 for the long term storage stable bendamustine-containing composition, as measured using USP monograph for polyethylene glycol; and
   iii) thioglycerol at a concentration of about 5 mg/mL; or
      IV. a) bendamustine or a pharmaceutically acceptable salt thereof; and
b) a pharmaceutically acceptable fluid including
   i) polyethylene glycol and propylene glycol;
   ii) sodium acetate in an amount sufficient to obtain a pH of from about 3.3 to about 4.2 for the long term storage stable bendamustine-containing composition, as measured using USP monograph for polyethylene glycol; and
   iii) a stabilizing amount of thioglycerol; or
      V. a) bendamustine or a pharmaceutically acceptable salt thereof at a concentration of about 25 mg/mL; and
b) a pharmaceutically acceptable fluid including
   i) 90% polyethylene glycol and 10% propylene glycol;
   ii) sodium acetate in an amount sufficient to obtain a pH of from about 3.3 to about 4.2 for the long term storage stable bendamustine-containing composition, as measured using USP monograph for polyethylene glycol; and
   iii) thioglycerol at a concentration of about 5 mg/mL; or
      VI. a) bendamustine or a pharmaceutically acceptable salt thereof at a concentration of about 25 mg/mL; and
b) a pharmaceutically acceptable fluid including
   i) 85% polyethylene glycol and 15% propylene glycol;
   ii) sodium acetate in an amount sufficient to obtain a pH of from about 3.3 to about 4.2 for the long term storage stable bendamustine-containing composition, as measured using USP monograph for polyethylene glycol; and
   iii) thioglycerol at a concentration of about 5 mg/mL.

Each of these compositions have the same stability profiles already described, i.e. having less than about 5% total esters, on a normalized peak area response (“PAR”) basis as determined by high performance liquid chromatography (“HPLC”) at a wavelength of 223 nm, after at least about 15 months of storage at a temperature of from about 5° C. to about 25° C.

Another embodiment of the invention provides methods of treating cancer in mammals. The methods include administering to a mammal in need thereof an effective amount of one of the bendamustine-containing compositions described herein. Since the active ingredient portion of the inventive composition is an FDA-approved drug, those of ordinary skill will recognize that the doses of bendamustine employed in this aspect of the invention will be similar to those employed in any treatment regimens designed for bendamustine as marketed under the trade name TREANDA. The patient package insert containing dosing information is incorporated herein by reference. The methods of treatment also include administering the inventive formulations for any purpose or physical condition for which bendamustine has been indicated as being useful.

Another embodiment of the invention includes methods of preparing bendamustine-containing compositions described herein. The methods include combining lyophilized bendamustine preferably as the HCl salt in a pharmaceutically acceptable fluid:
A) i) a mixture of PEG and PG within the desired ratios described herein, e.g. 90:10, etc.;
   ii) an organic compound or an inorganic compound in an amount sufficient to obtain a pH of from about 6.5 to about 11 for the polyethylene glycol, as measured using USP monograph for polyethylene glycol; and
   iii) a stabilizing amount of an antioxidant.

The steps are carried out under pharmaceutically acceptable conditions for sterility and manufacturing.

In a further aspect of the invention, there are provided methods of controlling or preventing the formation of polyethylene glycol esters and propylene glycol esters in bendamustine-containing compositions during long term storage. The methods include combining an amount of bendamustine or a pharmaceutically acceptable salt thereof with a sufficient amount of a pharmaceutically acceptable fluid containing:
i) a mixture of PEG and PG in the ratios described herein;
ii) an organic compound or an inorganic compound in an amount sufficient to obtain a pH of from about 6.5 to about 11 for the polyethylene glycol, as measured using USP monograph for polyethylene glycol; and
iii) a stabilizing amount of an antioxidant.

Further optional steps in accordance therewith include transferring one or more pharmaceutically acceptable doses of the formulations into a suitable sealable container and storing the sealed container at a temperature of from about 5° C. to about 25° C. As a result of carrying out these steps, it is possible to control or substantially prevent the formation of impurities which otherwise occur with bendamustine-containing compositions during long term storage so that the artisan is provided with bendamustine-containing formulations having less than about 5% total esters PAR as determined by HPLC at a wavelength of 223 nm, after at least about 15 months of storage at a temperature of from about 5° C. to about 25° C.

The compositions of the present invention can be packaged in any suitable sterile vial or container fit for the sterile storage of a pharmaceutical such as bendamustine. Preferably, the vials containing the formulation are sparged with nitrogen under seal before storage. Suitable containers can be glass vials, polypropylene or polyethylene vials or other special purpose containers and be of a size sufficient to hold one or more doses of bendamustine.

A further aspect of the invention includes kits containing lyophilized bendamustine or a pharmaceutically acceptable salt thereof in a first container or vial; and, in a second container, a sufficient amount of a pharmaceutically acceptable fluid such as those described herein:
i) a mixture of PEG and PG;
ii) an organic compound or an inorganic compound in an amount sufficient to obtain a pH of from about 6.5 to about 11 for the polyethylene glycol, as measured using USP monograph for polyethylene glycol; and
iii) a stabilizing amount of an antioxidant.

For purposes of this embodiment, the amount of fluid which is sufficient is an amount which allows the bendamustine to be dissolved or dispersed to a degree which renders the liquid composition ready for use, i.e. to administer to a patient in need thereof directly, or for dilution into a larger volume infusion at point of delivery.

As will be appreciated by those of ordinary skill, the kit will contain other pharmaceutically necessary materials for storing and/or administering the drug, including instructions for storage and use, additional diluents, if desired, etc.

### EXAMPLES

The following examples serve to provide further appreciation of the invention but are not meant in any way to restrict the effective scope of the invention.

### Comparative Example 1

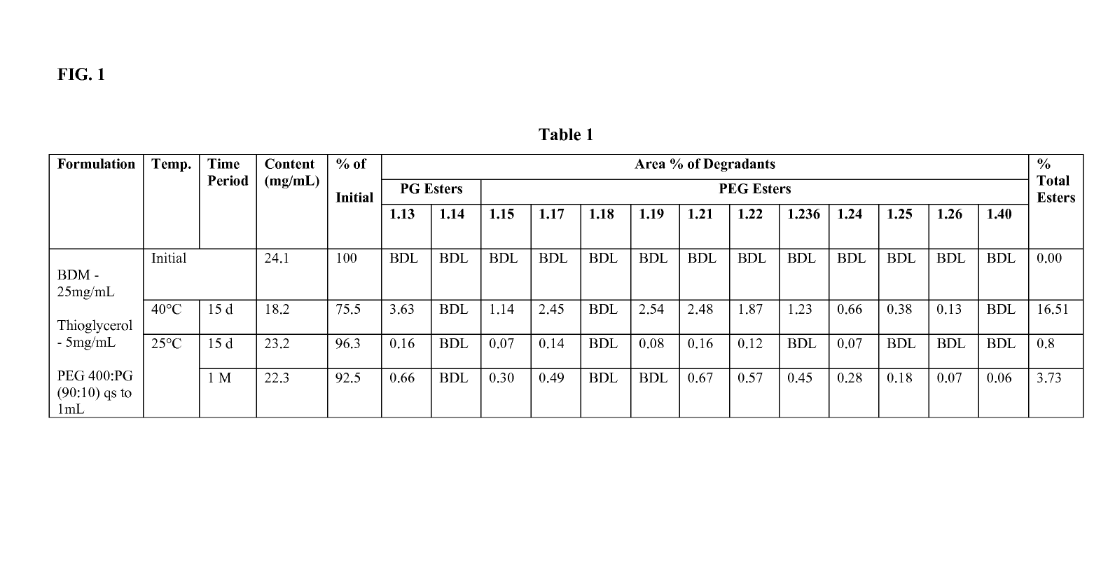

A mixture of PEG:PG (90:10) was prepared by combining 10 ml of PG with PEG 400 qs 100 ml. Thioglycerol at a concentration of 5 mg/ml was added to 80 ml of the PEG:PG (90:10) mixture and mixed well. The PEG:PG (90:10) and thioglycerol mixture was sparged with N₂. Bendamustine HCl at a concentration of 25 mg/ml was then added to 40 ml of the PEG:PG (90:10) and thioglycerol mixture, and mixed well. The volume of the bendamustine-containing formulation was made up to 50 ml with the PEG:PG (90:10) mixture, and then sparged with N₂. The bendamustine-containing formulation was then filtered and transferred to 5 cc vials, with each vial containing 4 ml. The vials were sparged with N₂, stoppered, crimped with aluminum seals. The samples were maintained at 40° C., 25° C. and 5° C. and analyzed after 15 days, one month, three months or five months for drug content and impurity profile as indicated in FIG. 1 (Table 1). The results obtained are presented in FIG. 1 (Table 1). At 14 days at 40° C., the pH of the bendamustine-containing formulation was taken in accordance with the USP official monograph. 5 g of the final bendamustine-containing formulation was added to 100 ml carbon dioxide free water, and 0.3 ml of saturated KCl solution was added. The pH was measured to be 3.38.

As shown in FIG. 1 (Table 1), the sample, which did not include NaOH, did not provide long term storage stability. This sample exhibited more than 16% total esters compared to initial after only 15 days at 40° C. It was determined that bendamustine-containing compositions with such high ester formation would not be suitable for long term storage. It was determined that the cause of the excess ester formation was the PEG.

### Example 2

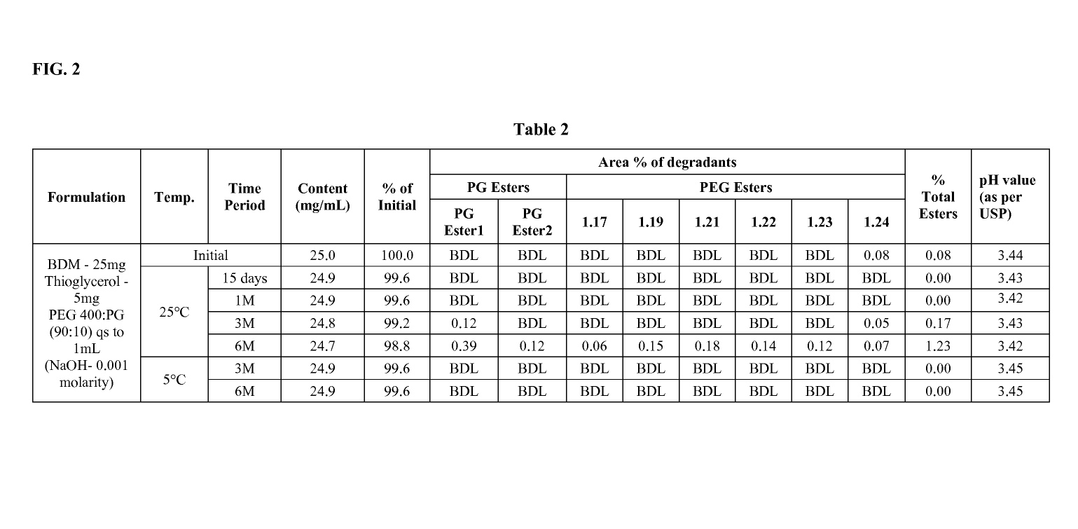

A mixture of PEG 400 treated with NaOH was prepared by combining 200 μl of 1N NaOH to a concentration of 0.001 molarity and PEG qs to 200 ml, and mixing well. The pH of the PEG 400 and NaOH mixture was taken in accordance with the USP official monograph. 5 g of the PEG 400 and NaOH mixture was added to 100 ml carbon dioxide free water, and 0.3 ml of saturated KCl solution was added. The pH was then measured to be 7.30, which is within the preferred range. A PEG:PG (90:10) mixture was prepared by combining 20 ml of PG and the PEG 400 and NaOH mixture qs 200 ml. Thioglycerol at a concentration of 5 mg/ml was added to 60 ml of the PEG:PG (90:10) mixture and mixed well. Bendamustine HCl at a concentration of 25 mg/ml was then added to 40 ml of the PEG:PG (90:10) and thioglycerol mixture, and mixed well. The volume of the bendamustine-containing formulation was made up to 75 ml with the PEG:PG (90:10) solution. The bendamustine-containing formulation was then filtered and transferred to 5 cc vials, with each vial containing 4 ml. The pH of the bendamustine-containing formulations was taken in accordance with the USP official monograph. 5 g of the final bendamustine-containing formulation was added to 100 ml carbon dioxide free water, and 0.3 ml of saturated KCl solution was added. The pH was then measured and recorded in FIG. 2 (Table 2). The vials were sparged with N₂, stoppered, crimped with aluminum seals. The samples were maintained at 25° C. and 5° C. and analyzed after 15 days, one month, three months, or six months for drug content, pH and impurity profile as indicated in FIG. 2 (Table 2). The results obtained are presented in FIG. 2 (Table 2).

As shown in FIG. 2 (Table 2), even without “pre” sparging steps, bendamustine, when dissolved in polyethylene glycol and propylene glycol, in the presence of a stabilizing amount of thioglycerol, and NaOH at a concentration of 0.001 molarity, had substantially no increase in total degradants after a period of at least six months at 25° C. The bendamustine-containing compositions had about 1.23% total esters after 6 months analysis at 25° C. Additionally, the pH of the compositions was maintained at about 3.4 throughout the duration of the long term storage. The data presented in FIG. 2 (Table 2) translates into bendamustine-containing compositions including PEG and PG, an antioxidant, and NaOH having a shelf life of at least about 15 months of storage at a temperature of from 5° C. to about 25° C. with levels of impurities within the levels required herein.

### Example 3

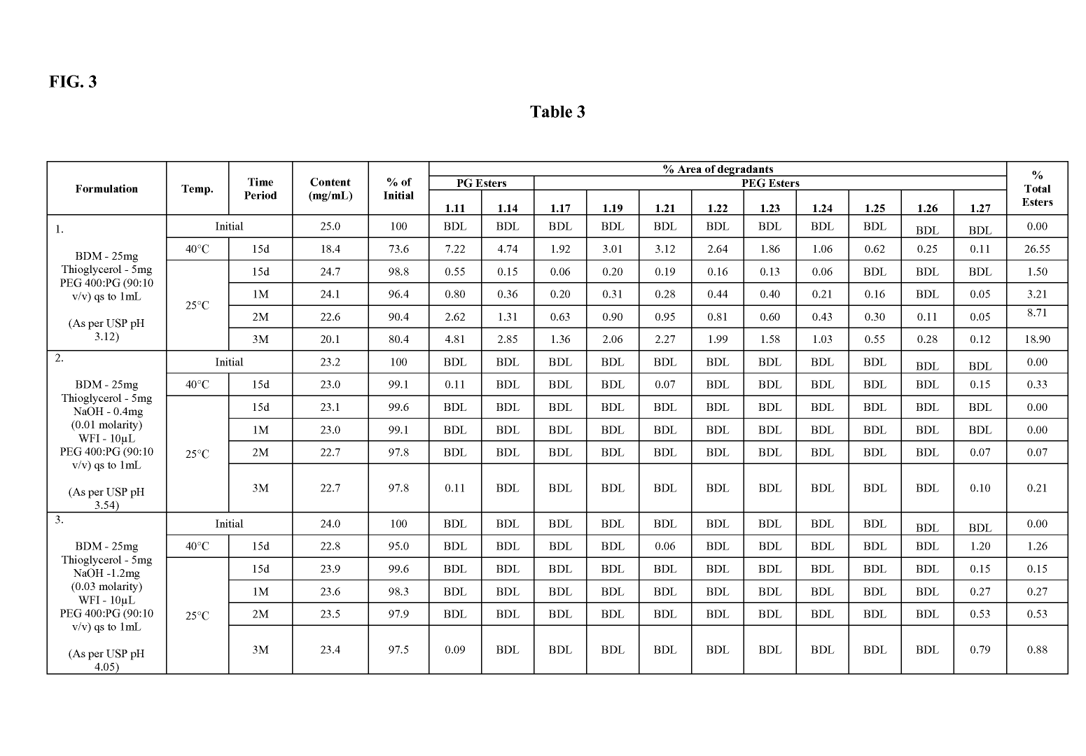

PEG:PG (90:10) mixtures were prepared by combining 10 ml of PG with PEG 400 qs 100 ml. Thioglycerol at a concentration of 5 mg/ml was added to 80 ml of the PEG:PG (90:10) mixture and mixed well. Bendamustine HCl at a concentration of 25 mg/ml was then added to 40 ml of the PEG:PG (90:10) and thioglycerol mixture, and mixed well. In addition to a sample, in which no NaOH was added (Sample 1), two samples were made in which a 1N NaOH solution was added to the PEG:PG (90:10) mixture to a concentration of 0.01 or 0.03 molarity (Samples 2 and 3, respectively), as indicated in FIG. 3 (Table 3), and mixed. The 0.01 and 0.03 molarity samples are unlike the samples in Examples 1 and 2, where the concentration of NaOH was 0.001 molarity. The volume of the bendamustine-containing solution was made up to 50 ml with the PEG:PG (90:10) mixture. The bendamustine-containing formulation was then filtered and transferred to 5 cc vials, with each vial containing 4 ml. The initial pH of the bendamustine-containing formulations was taken in accordance with the USP official monograph. 5 g of the final bendamustine-containing formulation was added to 100 ml carbon dioxide free water, and 0.3 ml of saturated KCl solution was added. The pH was then measured and recorded in FIG. 3 (Table 3). The vials were sparged with N₂, stoppered, crimped with aluminum seals. The samples were maintained at 40° C. and 25° C. and analyzed after 15 days, one month, two months, or three months for drug content and impurity profile as indicated in FIG. 3 (Table 3). The results obtained are presented in FIG. 3 (Table 3).

As shown in FIG. 3 (Table 3), bendamustine, when dissolved in polyethylene glycol and propylene glycol, in the presence of thioglycerol and NaOH at a concentration of 0.01 molarity or 0.03 molarity, has a pH of about 3.5 to about 4, which is within the preferred pH range. The bendamustine-containing samples according to the invention had substantially no increase in total degradants after a period of at least three months at 25° C. The bendamustine-containing compositions with NaOH concentration of 0.01 molarity and 0.03 molarity had about 0.33% and 1.26% total esters, respectively, after 15 days analysis at 40° C. This data supports the position that bendamustine-containing compositions according to the invention have a shelf life of at least about 2 years, if not longer, when stored under ambient or refrigerated storage conditions with levels of impurities within the levels required herein.

Also shown in FIG. 3 (Table 3), the control sample, which did not include NaOH did not provide long term storage stability. The pH of the control sample was 3.12. This sample exhibited more than 26% total esters compared to initial after only 15 days at 40° C., and almost 19% total esters compared to initial after 3 months at 25° C. Bendamustine-containing compositions with such high levels of degradation would not be long term storage stable.

### Example 4

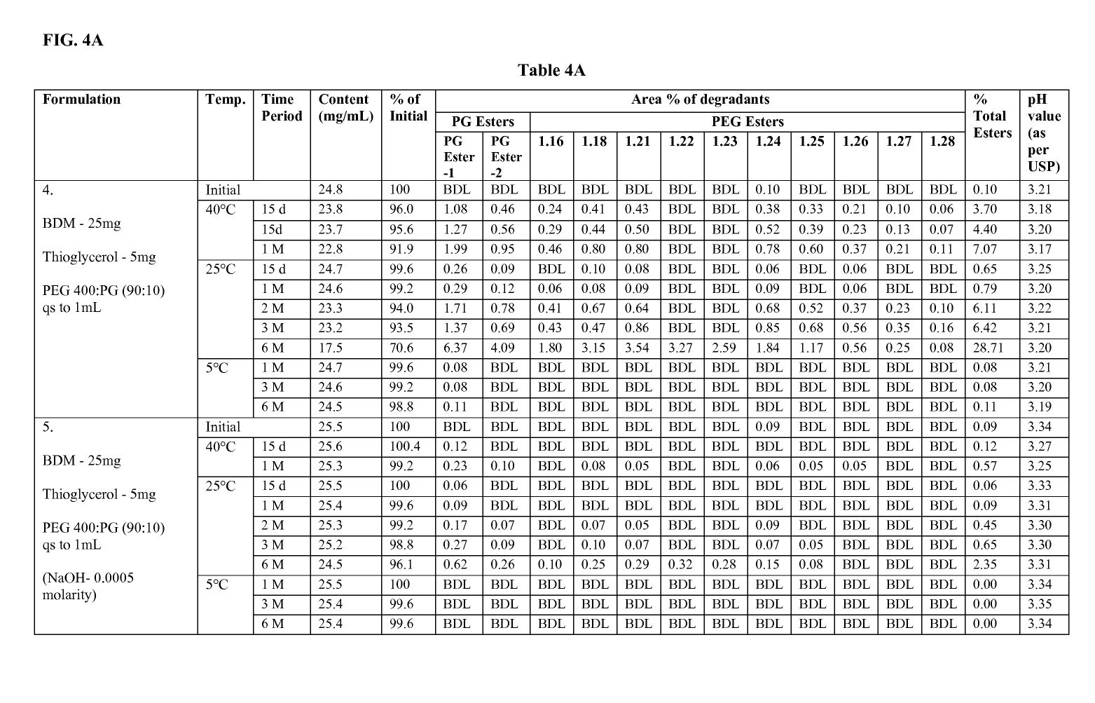

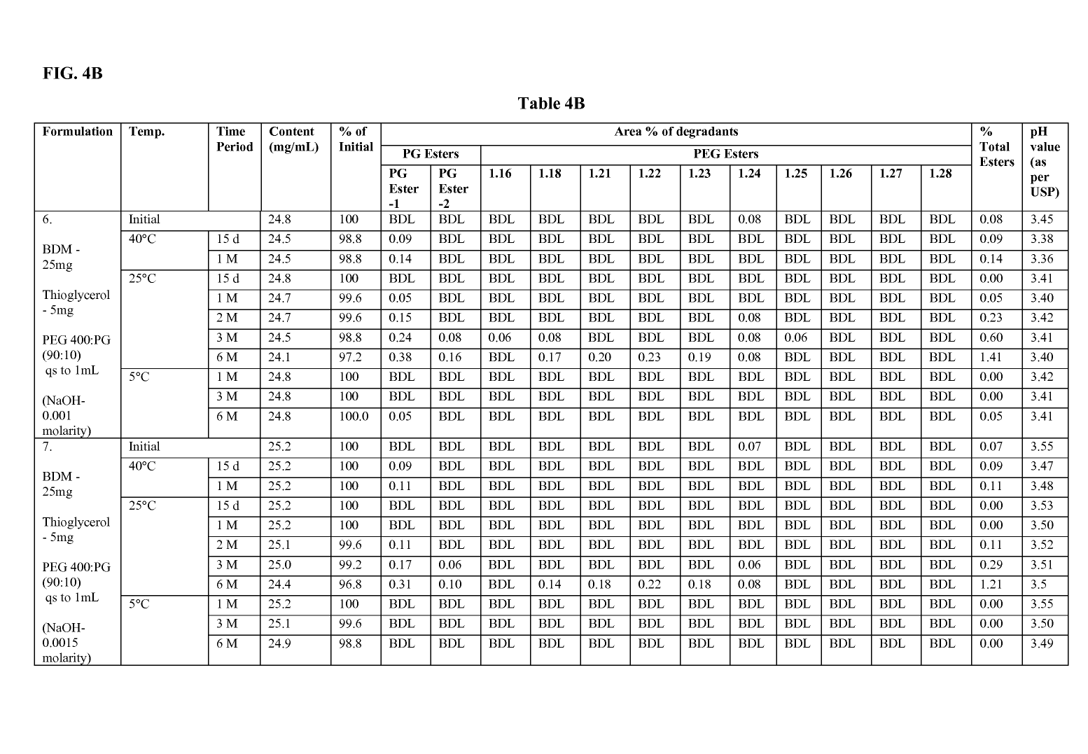

Mixtures of PEG 400 with NaOH were prepared by combining 0.1 ml, 0.2 ml or 0.3 ml (Samples 5, 6 and 7, respectively) of 1N NaOH and PEG qs to 200 ml, and mixing well. The pH of the PEG 400 and NaOH mixtures was taken in accordance with the USP official monograph. 5 g of the PEG 400 and NaOH mixtures were added to 100 ml carbon dioxide free water, and 0.3 ml of saturated KCl solution was added. The pH was then measured. The pH of the PEG 400 and NaOH mixture for Sample 5 was 6.32. The pH of the PEG 400 and NaOH mixture for Sample 6 was 7.30. The pH of the PEG 400 and NaOH mixture for Sample 7 was 7.89. The pH for the PEG 400 and NaOH mixtures for each of Samples 5, 6 and 7 were within the preferred range. Mixtures of PEG:PG (90:10) were prepared by combining 20 ml of PG with PEG 400 qs 200 ml, without NaOH (Sample 4) or with NaOH at a concentration of 0.0005, 0.001, or 0.0015 molarity (Samples 5, 6 and 7, respectively), as indicated in FIGS. 4A and 4B (Tables 4A and 4B). Thioglycerol at a concentration of 5 mg/ml was added to 80 ml of the PEG:PG (90:10) mixture and mixed well. Bendamustine HCl at a concentration of 25 mg/ml was then added to 80 ml of the PEG:PG (90:10) and thioglycerol mixture, and mixed well. The volume of the bendamustine-containing formulation was made up to 100 ml with the PEG:PG (90:10) mixture, and mixed. The bendamustine-containing formulation was then filtered and transferred to 5 cc vials, with each vial containing 4 ml. The vials were sparged with N₂, stoppered, crimped with aluminum seals. The samples were maintained at 40° C., 25° C. and 5° C. and analyzed after 15 days, one month, two months, three months or six months for drug content, impurity profile and pH as indicated in FIGS. 4A and 4B (Tables 4A and 4B). The pH was evaluated as per the USP official monograph. 5 g of the final bendamustine-containing formulation was added to 100 ml carbon dioxide free water, and 0.3 ml of saturated KCl solution was added. The pH was then measured. The results obtained are presented in FIGS. 4A and 4B (Tables 4A and 4B).

As shown in FIGS. 4A and 4B (Tables 4A and 4B), bendamustine, when dissolved in polyethylene glycol and propylene glycol, in the presence of thioglycerol and NaOH at a concentration of 0.0005 molarity, 0.001 molarity or 0.0015 molarity, the bendamustine-containing samples according to the invention have a pH of about 3.3 to about 3.6. This is within the preferred pH range. The bendamustine-containing samples according to the invention had no or substantially no increase in total degradants after a period of at least six months at 5° C. The bendamustine-containing compositions with NaOH concentration of 0.005 molarity had about 2.35% total esters after six months analysis at 25° C. The bendamustine-containing compositions with NaOH concentration of 0.001 molarity had about 1.41% total esters after six months analysis at 25° C. The bendamustine-containing compositions with NaOH concentration of 0.0015 molarity had about 1.21% total esters after six months analysis at 25° C. This data projects a shelf life of at least about 2 years, if not longer, when stored under ambient or refrigerated storage conditions with levels of impurities within the levels required herein.

Also shown in FIGS. 4A and 4B (Tables 4A and 4B), the control sample, which did not include NaOH, did not provide long term storage stability. The pH of the control sample is ranges from 3.17 to 3.25. This sample exhibited more than 28% total esters compared to initial after six months at 25° C. Bendamustine-containing compositions with such high levels of degradation would not be long term storage stable.

### Example 5

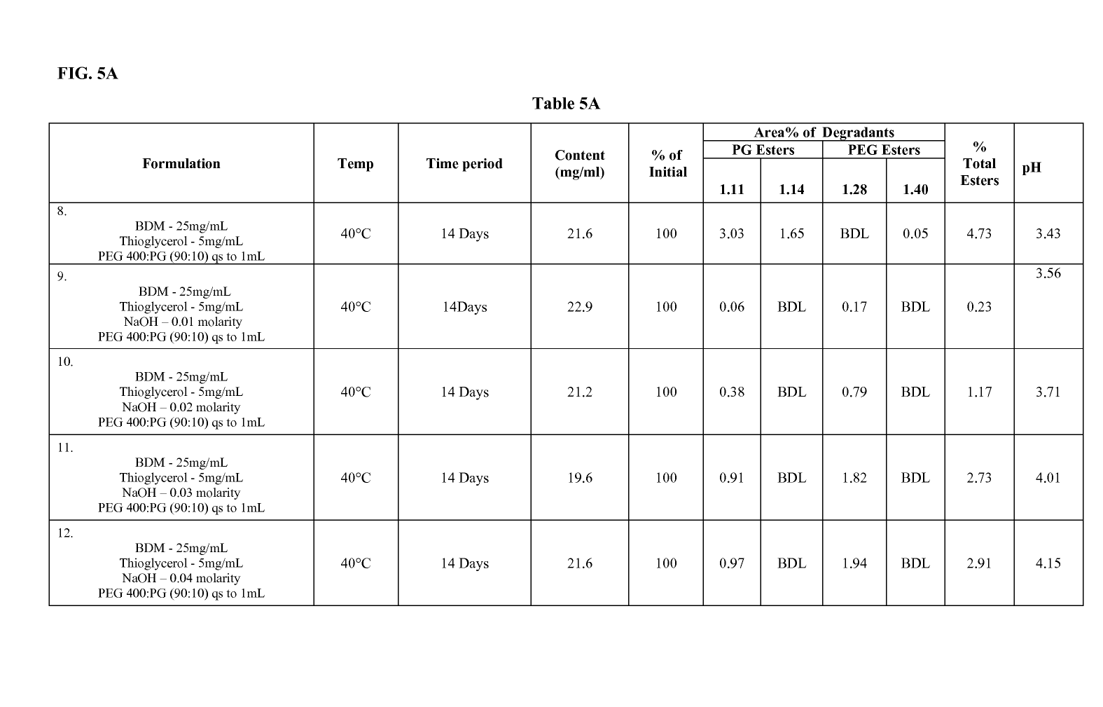

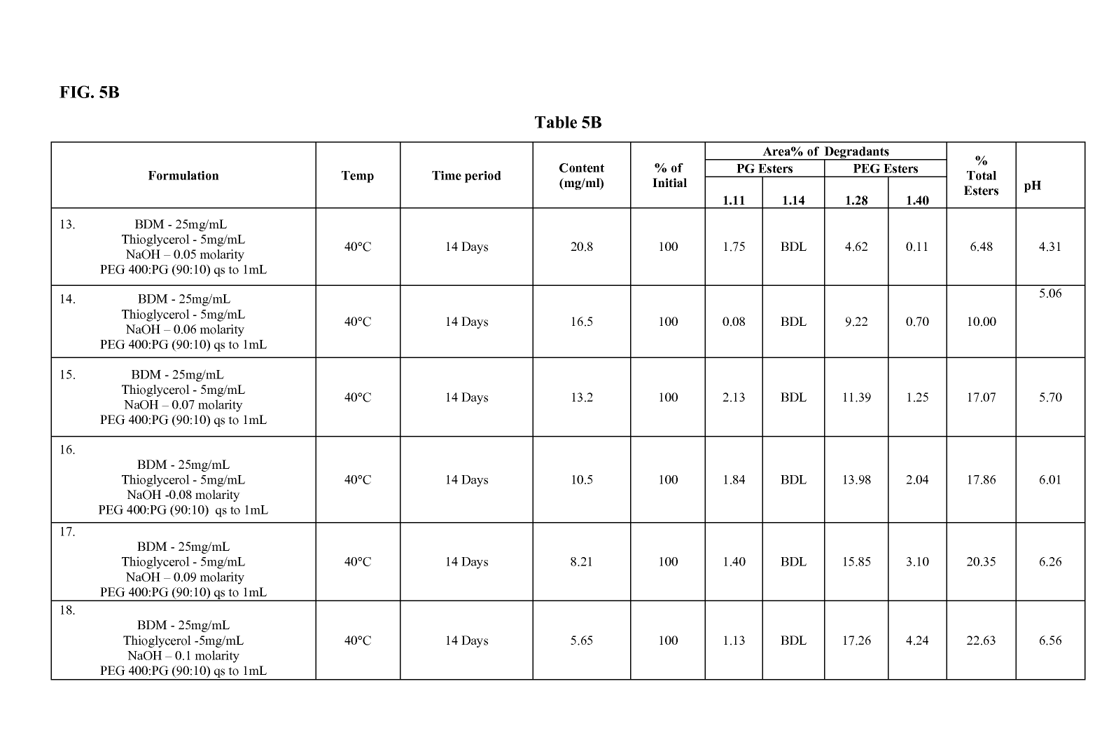

PEG:PG (90:10) mixtures were prepared by combining 10 ml of PG and PEG 400 qs 100 ml. Thioglycerol at a concentration of 5 mg/ml was added to 50 ml of the PEG:PG (90:10) mixture and mixed well. Bendamustine HCl at a concentration of 25 mg/ml was then added to 50 ml of the PEG:PG (90:10) and thioglycerol mixture, and mixed well. The volume of the bendamustine-containing formulation was made up to 60 ml with the PEG:PG (90:10) solution. The bendamustine-containing formulation was transferred to 5 cc vials, with each vial containing 5 ml. Unlike in previous Examples, to each vial, except the control which was without NaOH (Sample 8), a 1N NaOH solution was added, yielding a final NaOH concentration of 0.01, 0.02, 0.03, 0.04, 0.05, 0.06, 0.07, 0.08, 0.09 or 0.1 molarity (Samples 9, 10, 11, 12, 13, 14, 15, 16, 17 and 18, respectively), as indicated in FIGS. 5A and 5B (Tables 5A and 5B). The vials were sparged with N₂, stoppered, crimped with aluminum seals. The samples were maintained at 40° C. and analyzed after 14 days for drug content and impurity profile as indicated in FIGS. 5A and 5B (Tables 5A and 5B). The results obtained are presented in FIGS. 5A and 5B (Tables 5A and 5B).

As shown in FIGS. 5A and 5B (Tables 5A and 5B), bendamustine, when dissolved in polyethylene glycol and propylene glycol, in the presence of thioglycerol and NaOH at a concentration of 0.01 molarity, 0.02 molarity, 0.03 molarity, or 0.04 molarity, the bendamustine-containing samples according to the invention have substantially low amount of total degradants after a period of about 14 days at 40° C. compared to bendamustine-containing samples having no NaOH and NaOH at a concentration 0.05 molarity or greater. The bendamustine-containing compositions with NaOH concentration from 0.01 to 0.04 molarity had from 0.23% to 2.91% total esters after 14 days analysis at 40° C. This data projects a shelf life of at least about 2 years, if not longer, when stored under ambient or refrigerated storage conditions with levels of impurities within the levels required herein.

Also shown in FIGS. 5A and 5B (Tables 5A and 5B), the control sample, which did not include NaOH, did not provide long term storage stability. This sample exhibited more than 4% total esters after 14 days at 40° C. These bendamustine-containing compositions with such high levels of degradation would not be long term storage stable.

The bendamustine-containing compositions with NaOH concentration of 0.05 molarity or greater had more than 6% total esters after 14 days analysis at 40° C. These bendamustine-containing compositions with such high levels of degradation would not be long term storage stable.

### Example 6

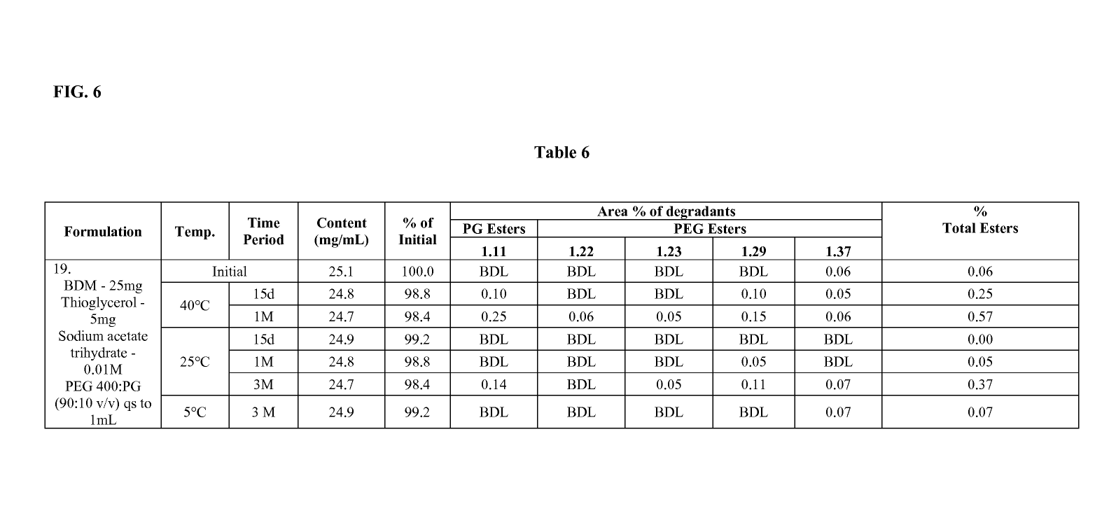

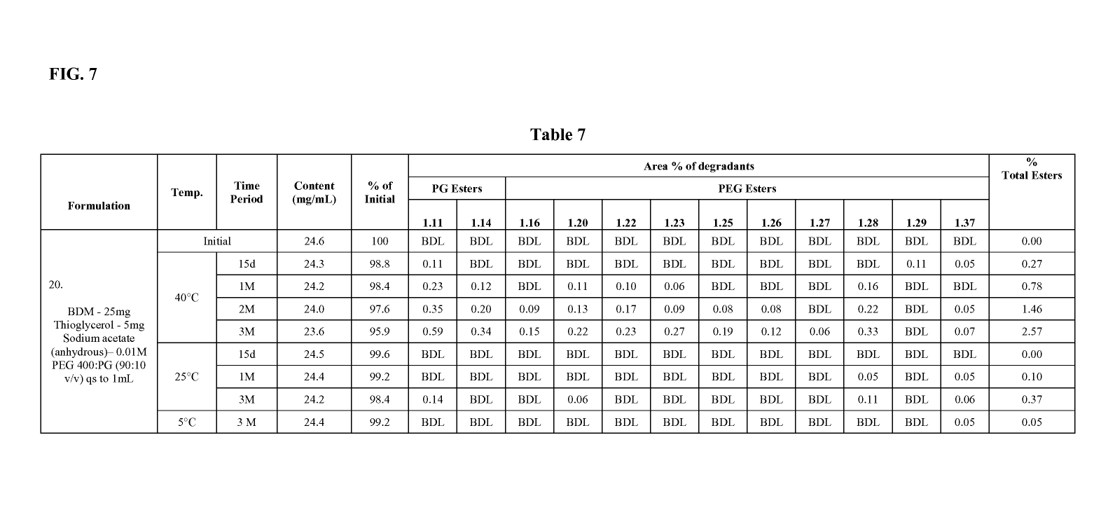

PEG and sodium acetate mixtures were prepared by adding sodium acetate (sodium acetate trihydrate (Sample 19) in FIG. 6 (Table 6) and sodium acetate anhydrous (Sample 20) in FIG. 7 (Table 7)) at a concentration of 0.01 molarity to 81 mL PEG 400 and mixing. The pH was evaluated as per the USP official monograph. 5 g of the PEG and sodium acetate mixture was added to 100 ml carbon dioxide free water, and 0.3 ml of saturated KCl solution was added. The pH was then measured. The PEG and sodium acetate mixture of Sample 19 had a pH of 3.74 and the PEG and sodium acetate mixture of Sample 20 had a pH of 3.67. The PEG and sodium acetate mixtures of both Samples 19 and 20 are within the preferred range. PEG:PG (90:10) and sodium acetate mixtures were prepared by combining 10 ml of PG with the PEG 400 sodium acetate mixture and mixing. Thioglycerol at a concentration of 5 mg/ml was added to the PEG:PG (90:10) sodium acetate solution and mixed. Bendamustine HCl at a concentration of 25 mg/ml was then added to the PEG:PG (90:10) sodium acetate and thioglycerol mixture, and mixed. The volume of the bendamustine-containing formulation was made up to 100 ml with PEG 400. The bendamustine-containing formulation was then filtered and transferred to 5 cc vials, with each vial containing 4 ml. The vials were sparged with N₂, stoppered, crimped with aluminum seals. The samples were maintained at 40° C., 25° C. and 5° C. and analyzed after 15 days, one month, or three months for drug content and impurity profile as indicated in FIGS. 6 and 7 (Tables 6 and 7). The results obtained are presented in FIGS. 6 and 7 (Tables 6 and 7).

As shown in FIGS. 6 and 7 (Tables 6 and 7), bendamustine, when dissolved in polyethylene glycol and propylene glycol, in the presence of thioglycerol and sodium acetate at a concentration of 0.01M, the bendamustine-containing samples according to the invention have substantially low amount of total degradants after a period of about 15 days at 40° C. The bendamustine-containing compositions with sodium acetate concentration of 0.01M also had substantially no degradants after three months analysis at 25° C. This data projects a shelf life of at least about 2 years, if not longer, when stored under ambient or refrigerated storage conditions with levels of impurities within the levels required herein.

### Example 7

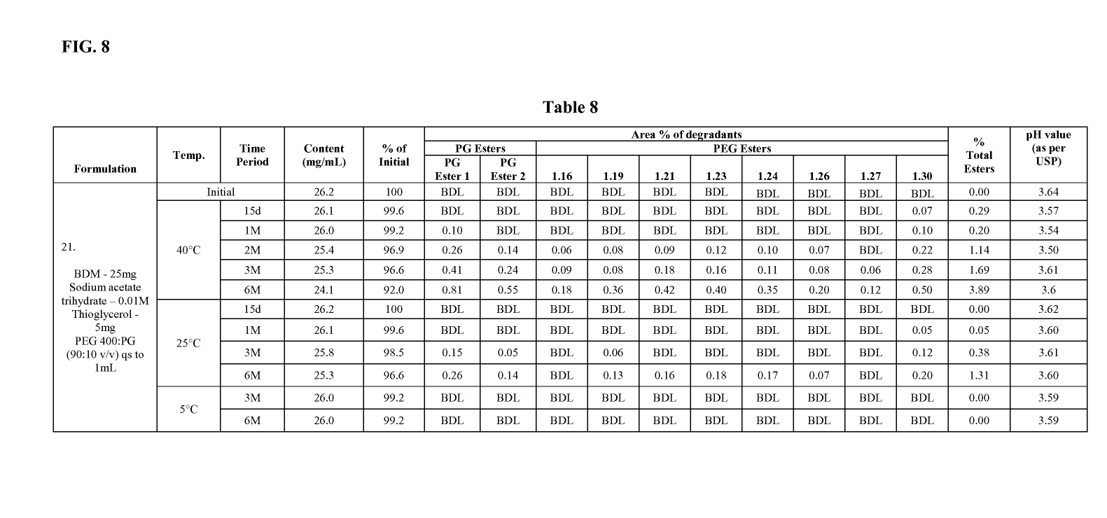

A PEG sodium acetate mixture was prepared by adding sodium acetate trihydrate at a concentration of 0.01 molarity to 81 mL PEG 400, mixing. The pH was evaluated as per the USP official monograph. 5 g of the PEG and sodium acetate mixture was added to 100 ml carbon dioxide free water, and 0.3 ml of saturated KCl solution was added. The pH was then measured. The PEG and sodium acetate mixture had a pH of 3.74, which is within the preferred range. A PEG:PG (90:10) sodium acetate mixture was prepared by combining 10 ml of PG with the PEG 400 sodium acetate mixture and mixing. Thioglycerol at a concentration of 5 mg/ml was added to the PEG:PG (90:10) sodium acetate mixture and mixed. Bendamustine HCl at a concentration of 25 mg/ml was then added to the PEG:PG (90:10) sodium acetate and thioglycerol mixture, and mixed. The volume of the bendamustine-containing formulation was made up to 100 ml with PEG 400. The bendamustine-containing formulation (Sample 21) was then filtered and transferred to 5 cc vials, with each vial containing 4 ml. The vials were sparged with N₂, stoppered, crimped with aluminum seals. The samples were maintained at 40° C., 25° C. and 5° C. and analyzed after 15 days, one month, or three months for drug content, impurity profile and pH as indicated in FIG. 8 (Table 8). The pH was evaluated as per the USP official monograph. 5 g of the final bendamustine-containing formulation was added to 100 ml carbon dioxide free water, and 0.3 ml of saturated KCl solution was added. The pH was then measured. The results obtained are presented in FIG. 8 (Table 8).

As shown in FIG. 8 (Table 8), bendamustine, when dissolved in polyethylene glycol and propylene glycol, in the presence of thioglycerol and sodium acetate at a concentration of 0.01M, the bendamustine-containing samples according to the invention have a pH of about 3.5 to about 3.64. This is within the preferred pH range. The bendamustine-containing samples according to the invention have substantially low amount of total degradants after a period of about six months at 25° C. The bendamustine-containing compositions with sodium acetate concentration of 0.01M also had substantially no degradants after six months analysis at 5° C.

The area % of the total esters increased about 1.31% over six months storage at 25° C. Such an increase projects a shelf life of at least about 2 years, if not longer, when stored under ambient or refrigerated storage conditions with levels of impurities within the levels required herein.

We claim:

1. A long term storage stable bendamustine-containing composition, comprising:
a) bendamustine or a pharmaceutically acceptable salt thereof; and
b) a pharmaceutically acceptable fluid comprising
i) a mixture of polyethylene glycol, and propylene glycol;
ii) an organic compound or an inorganic compound in an amount sufficient to obtain a pH of from about 6.0 to about 11 for the polyethylene glycol, as measured using USP monograph for polyethylene glycol; and
iii) a stabilizing amount of an antioxidant;
said bendamustine-containing composition having less than about 5% total polyethylene glycol esters and propylene glycol esters, on a normalized peak area response (“PAR”) basis as determined by high performance liquid chromatography (“HPLC”) at a wavelength of 223 nm, after at least about 15 months of storage at a temperature of from about 5° C. to about 25° C.

2. The long term storage stable bendamustine-containing composition of claim 1, wherein the amount of the organic compound or an inorganic compound is provided in an amount sufficient to obtain a pH of from about 6.5 to about 8 for the polyethylene glycol, as measured using USP monograph for polyethylene glycol.

3. The long term storage stable bendamustine-containing composition of claim 1, wherein the pharmaceutically acceptable fluid comprises an inorganic compound selected from the group consisting of salts of hydroxides and salts of phosphates.

4. The long term storage stable bendamustine-containing composition of claim 3, wherein the inorganic compound is sodium hydroxide.

5. The long term storage stable bendamustine-containing composition of claim 1, wherein the pharmaceutically acceptable fluid comprises an organic compound selected from the group consisting of carboxylic compounds, nitrogenous compounds, carbonates, and salts thereof.

6. The long term storage stable bendamustine-containing composition of claim 5, wherein the organic compound is sodium acetate or is diethanolamine.

7. The long term storage stable bendamustine-containing composition of claim 1, wherein the bendamustine concentration is from about 20 mg/mL to about 60 mg/mL.

8. The long term storage stable bendamustine-containing composition of claim 7, wherein the bendamustine concentration is from about 25 mg/mL to about 50 mg/mL.

9. The long term storage stable bendamustine-containing composition of claim 8, wherein the bendamustine concentration is about 25 mg/mL.

10. The long term storage stable bendamustine-containing composition of claim 1, wherein the pharmaceutically acceptable fluid comprises about 90% polyethylene glycol and about 10% propylene glycol.

11. The long term storage stable bendamustine-containing composition of claim 1, wherein the pharmaceutically acceptable fluid comprises about 85% polyethylene glycol and about 15% propylene glycol.

12. The long term storage stable bendamustine-containing composition of claim 1, wherein the antioxidant is selected from the group consisting of thioglycerol, monothioglycerol, lipoic acid, propyl gallate, methionine, cysteine, metabisulfites, sodium formaldehyde sulfoxylate, phenol-containing aromatic and aliphatic compounds and dihydrolipoic acid.

13. The long term storage stable bendamustine-containing composition of claim 12, wherein the antioxidant is thioglycerol or monothioglycerol.

14. The long term storage stable bendamustine-containing composition of claim 1, wherein the stabilizing amount of the antioxidant is from about 2.5 mg/mL to about 35 mg/mL.

15. The long term storage stable bendamustine-containing composition of claim 14, wherein the stabilizing amount of the antioxidant is from about 5 mg/mL to about 20 mg/mL.

16. The long term storage stable bendamustine-containing composition of claim 15, wherein the stabilizing amount of the antioxidant is about 5 mg/mL.

17. The long term storage stable bendamustine-containing composition of claim 1, wherein the concentration of the inorganic compound is from about 0.0005 molarity to about 0.04 molarity.

18. The long term storage stable bendamustine-containing composition of claim 17, wherein the concentration of the inorganic compound is about 0.01 molarity.

19. The long term storage stable bendamustine-containing composition of claim 1, wherein the concentration of the organic compound is from about 0.005M to about 0.1M.

20. The long term storage stable bendamustine-containing composition of claim 19, wherein the concentration of the organic compound is about 0.01M.

21. The long term storage stable bendamustine-containing composition of claim 1, wherein the pH of the polyethylene glycol, as measured using the USP monograph for polyethylene glycol, is about 6.5 or about 8.

22. The long term storage stable bendamustine-containing composition of claim 1, wherein the pH of the long term storage stable bendamustine-containing composition, as measured using the USP monograph for polyethylene glycol, is from about 3.3 to about 4.

23. The long term storage stable bendamustine-containing composition of claim 22, wherein the pH of the long term storage stable bendamustine-containing composition, as measured using the USP monograph for polyethylene glycol, is about 3.5.

24. The long term storage stable bendamustine-containing composition of claim 1, wherein the amount of total polyethylene glycol esters and propylene glycol esters is less than about 3%.

25. The long term storage stable bendamustine-containing composition of claim 1, wherein the amount of total polyethylene glycol esters and propylene glycol esters is less than about 2.4%.

26. The long term storage stable bendamustine-containing composition of claim 1, wherein the amount of individual polyethylene glycol esters less than about 0.2% and individual propylene glycol esters is less than about 1.5%.

27. The long term storage stable bendamustine-containing composition of claim 1, wherein the amount of total polyethylene glycol esters is less than about 2%.

28. The long term storage stable bendamustine-containing composition of claim 1, wherein the amount of total propylene glycol esters is less than about 3%.

29. The long term storage stable bendamustine-containing composition of claim 1, wherein said long term storage is at least about 2 years.

30. A long term storage stable bendamustine-containing composition, comprising:
a) bendamustine or a pharmaceutically acceptable salt thereof; and
b) a pharmaceutically acceptable fluid comprising
i) 90% polyethylene glycol and 10% propylene glycol;
ii) sodium hydroxide in an amount sufficient to obtain a pH of from about 6.5 for the polyethylene glycol, as measured using the USP monograph for polyethylene glycol; and
iii) thioglycerol at a concentration of about 5 mg/mL;
said bendamustine-containing composition having less than about 5% total polyethylene glycol esters and propylene glycol esters, on a normalized peak area response (“PAR”) basis as determined by high performance liquid chromatography (“HPLC”) at a wavelength of 223 nm, after at least about 15 months of storage at a temperature of from about 5° C. to about 25° C.

31. A long term storage stable bendamustine-containing composition, comprising:
a) bendamustine or a pharmaceutically acceptable salt thereof at a concentration of about 25 mg/mL; and
b) a pharmaceutically acceptable fluid comprising
i) 90% polyethylene glycol and 10% propylene glycol;
ii) sodium acetate in an amount sufficient to obtain a pH of about 6.5 for the polyethylene glycol, as measured using the USP monograph for polyethylene glycol; and
iii) thioglycerol at a concentration of about 5 mg/mL;
said bendamustine-containing composition having less than about 5% total polyethylene glycol esters and propylene glycol esters, on a normalized peak area response (“PAR”) basis as determined by high performance liquid chromatography (“HPLC”) at a wavelength of 223 nm, after at least about 15 months of storage at a temperature of from about 5° C. to about 25° C.

32. A long term storage stable bendamustine-containing composition, comprising:
a) bendamustine or a pharmaceutically acceptable salt thereof; and
b) a pharmaceutically acceptable fluid comprising
i) a mixture of polyethylene glycol, and propylene glycol;
ii) an organic compound or an inorganic compound in an amount sufficient to obtain a pH of from about 3.3 to about 4.2 for the long term storage stable bendamustine-containing composition, as measured using the USP monograph for polyethylene glycol; and
iii) a stabilizing amount of an antioxidant;
said bendamustine-containing composition having less than about 5% total polyethylene glycol esters and propylene glycol esters, on a normalized peak area response (“PAR”) basis as determined by high performance liquid chromatography (“HPLC”) at a wavelength of 223 nm, after at least about 15 months of storage at a temperature of from about 5° C. to about 25° C.

33. A method of treating cancer in mammals, comprising administering an effective amount of a long term storage stable bendamustine-containing composition of claim 1 to a mammal in need thereof.
